SEQUENCE LISTING (3 sequences)

SEQ ID NO: 1 - RNA, 1479 nt, synthetic construct
  source 1..1479: mol_type = other RNA
aactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgcaccaaaagagaactgcaatgtttcaggacccacaggagcgaagcggcaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtaggcgataaatgtttaaagttttattctaaaattagtgagtatagacattattgttatagtttgtatggaacaacattagaacagcaatacaacaaaccgttgtgtgatttgttaattaggggcattaactgtcaaaagccactgtgtcctgaagaaaagcaaagacatctggacaaaaagcaaagattccataatataaggggtcggtggaccggtcgatgtatgtcttgttgcagatcatcaagaacacgtggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 2 - RNA, 1314 nt, synthetic construct
  source 1..1314: mol_type = other RNA
aactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgcatggcgacacaccaacacttcacgaatatatgctggatttgcagcctgaaaccaccgatctgtacggctatggccagctgaatgattcttctgaagaagaagatgaaattgatggtcctgctggacaagcagaacctgatagagcccactacaacattgtgacattttgctgcaaatgtgattcaacccttagactttgtgttcagtccacacatgttgacataagaactctggaagatctgctgatgggaactcttggaattgtgggccccatttgctcacagaaacctggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 3 - RNA, 1117 nt, synthetic construct
  source 1..1117: mol_type = other RNA
gggaaataagagagaaaagaagagtaagaagaaatataagagccaccatgagagtgatggcccccagaaccctgatcctgctgctgtctggcgccctggccctgacagagacatgggccggaagcggcggctctggaggaggcggctccggaggcatgcatggcgacacaccaacacttcacgaatatatgctggatttgcagcctgaaaccaccgatctgtacggctatggccagctgaatgattcttctgaagaagaagatgaaattgatggtcctgctggacaagcagaacctgatagagcccactacaacattgtgacattttgctgcaaatgtgattcaacccttagactttgtgttcagtccacacatgttgacataagaactctggaagatctgctgatgggaactcttggaattgtgggccccatttgctcacagaaacctggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaatgataataggctggagcctcggtggccatgcttcttgccccttgggcctccccccagcccctcctccccttcctgcacccgtacccccgtggtctttgaataaagtctgaaaaaaaaaaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0263633 A1
(43) Pub. Date: Sep. 10, 2026

(54) MIXED NANO-LIPID DELIVERY SYSTEM FOR mRNA, PREPARATION METHOD THEREFOR AND USE THEREOF

(71) Applicant: Beijing Dama Fangcheng Biotechnology Co., Ltd, Daxing District, Beijing (CN)

(72) Inventors: Zhenjun YANG, Haidian District, Beijing (CN); Jing YU, Haidian District, Beijing (CN); Fang WANG, Daxing District, Beijing (CN); Zhengdi XU, Daxing District, Beijing (CN)

(21) Appl. No.: 19/162,865

(22) PCT Filed: Nov. 9, 2023
(86) PCT No.: PCT/CN2023/130614

§ 371 Date: Sep. 5, 2025

(30) Foreign Application Priority Data
Mar. 8, 2023 (CN) 202310256877.6

(51) Int. Cl. A61K 48/00; A61K 9/1272; A61K 9/1277; A61K 9/51; A61K 31/7105; A61K 47/02; C12N 15/88

(52) U.S. Cl. CPC A61K 48/0041; A61K 9/1272; A61K 9/1277; A61K 9/5123; A61K 9/5192; A61K 31/7105; A61K 47/02; A61K 48/0075; C12N 15/88

(57) ABSTRACT

Disclosed are a mixed nano-lipid delivery system for mRNA, and a preparation method therefor and a use thereof. The delivery system is composed of an anionic and cationic mixed lipid, PEG2000-DSPE, a buffer system and mRNA; the anionic and cationic mixed lipid is composed of anionic nucleoside phospholipid TPS or CPS and cationic peptide lipid CLD or CLDA; and the buffer system is a PBS or Opti-MEM™ buffer system containing Ca2+ having a concentration 0.1 mM. The delivery system can efficiently deliver mRNA into cells and mice, and can realize long-time stable expression of proteins in vivo. In addition, an HPV E7 mRNA vaccine provided by the present disclosure can successfully activate humoral and cellular immunity in mice, reduce the mortality rate of HPV-related cervical cancer mice, and have good safety.

### TECHNICAL FIELD

The present disclosure relates to an anionic/cationic mixed lipid-based delivery system for encapsulating mRNA nucleic acid drugs, a preparation method therefor and a use thereof. The present disclosure belongs to the field of biomedical technology.

### BACKGROUND

Cervical cancer is the fourth most common cancer among women worldwide and the most prevalent gynecological malignancy in developing countries. Numerous etiological and epidemiological studies have shown that infection with high-risk human papillomavirus (HPV) is the primary cause of the development and progression of cervical cancer, including types 16, 18, 31, 33, 35, 39, 45, 51, 52, 56, 58, 59, 68, and 73. Among these, HPV 16 and 18 are the primary etiological factors, accounting for nearly 71% of cervical cancers globally. Long-term persistent viral infection leads to the continuous expression of HPV E6 and E7 oncoproteins, which significantly disrupt the cell growth cycle, impair DNA repair, and ultimately result in cancer.

Currently, there are three prophylactic HPV vaccines: Cervarix®, Gardasil®, and Gardasil® 9, which can prevent HPV infection in order to prevent HPV-related cancers. Although HPV vaccines are safe and effective and can provide long-term protection against HPV infection, they cannot eliminate existing infections or trigger cell-mediated immune responses to clear pre-existing lesions or even malignancies. HPV infection and the development of HPV-related cancers will continue to be a public health concern. Therefore, the development of therapeutic HPV vaccines is particularly important for the treatment of HPV-related cancers, especially cervical cancer.

Therapeutic vaccine strategies aim to combat precancerous or cancerous lesions by triggering cell-mediated immunity against HPV-infected cells. Therefore, HPV E6 and E7 proteins, which are specifically highly expressed in infected and cancerous cells, are ideal target antigens. Current therapeutic HPV vaccines include viral vector vaccines, bacterial vector vaccines, peptide/protein vaccines, nucleic acid (DNA and RNA) vaccines, dendritic cell-based vaccines, and tumor cell-based vaccines. Among them, nucleic acid vaccines represent a significant advancement in vaccine technology, offering flexibility and precision in antigen design, enabling rapid large-scale production in vitro. Compared with DNA vaccines, RNA vaccines have a single-stranded structure, are simpler and faster to synthesize, easier to introduce into cells, and the mRNA that has performed its function will then degrade without causing other toxic or side effects to the human body. Therefore, interest and research activities in mRNA vaccines are increasing. After vaccination, the protein synthesis system of human cells will synthesize the tumor-specific antigen protein encoded by mRNA, which acts as a “target” to induce the body's immune response against the “target”, triggering humoral and cytotoxic T cell responses to specifically attack tumor cells. In addition, mRNA-based vaccines allow direct processing of antigens through the endogenous major histocompatibility complex (MHC) class I antigen presentation pathway, making them an ideal choice for promoting the production of (CD8+ T cells), a key component of immune therapeutic responses against intracellular pathogens and cancers.

Since naked mRNA is easily degraded by nucleases in vivo, has a large molecular weight and is negatively charged, it cannot cross cell membranes on its own. Therefore, RNA must be encapsulated in a certain form to realize its full potential. Currently, common delivery methods include physical methods (microinjection, electroporation, hydrodynamic injection, etc.), viral vectors, and chemical carriers (cationic liposomes, lipoplexes, gold nanoparticles, etc.). Among them, physical methods are often difficult to operate, cause significant damage to cells, and are difficult to perform transfection on a large scale or in vivo. Although viral vectors exhibit high delivery efficiency and can avoid the risk of viral DNA integration into the host genome through flexible vector selection, components such as viral envelopes still have certain immunogenic risks, are easily cleared by the body during the second administration, and the production process of viral vectors is costly and time-consuming, which needs further research to solve. Chemical delivery methods such as liposomes have been widely used in in vivo and in vitro delivery due to their low cost and relatively simple encapsulation procedures. In all cases, the carrier must traverse the target cell membrane and, after cellular uptake, achieve endosomal escape to release the encapsulated nucleic acids internally. Only then can the encapsulated nucleic acids function at the correct site; otherwise, the substances will be degraded by enzymes in the lysosomes and lose activity.

Despite the aforementioned challenges, the advantages of liposomes and lipid complexes remain evident, such as causing less cellular damage than electroporation and posing a lower immunogenicity risk compared to viral vectors. Therefore, liposome- and lipid complex-mediated delivery remains a hot topic in current gene delivery vector research. In clinical and preclinical studies of RNA vaccines, cationic liposomes are often used for delivery. Cationic lipids utilize their electropositive characteristics to bind with negatively charged nucleic acid molecules through electrostatic interactions, forming liposomes that encapsulate the nucleic acids or producing lipid complexes with other structures. Many laboratories have successfully applied cationic liposome-transfected mRNA in studies against various pathogens such as rabies virus, Zika virus, influenza virus, etc. However, cationic liposomes exhibit high toxicity and readily bind to negatively charged serum proteins under physiological conditions, leading to immunogenicity and liver toxicity. These defects limit the further application of cationic liposomes in the encapsulation of mRNA for drug development.

The inventor(s) previously designed and synthesized anionic nucleobase lipids TPS/CPS (CN113583069A), which can bind and encapsulate single-stranded nucleic acid drugs through hydrogen bonding and n-n stacking interactions due to their nucleobase-containing head groups. By combining TPS/CPS with the previously designed and synthesized cationic lipids CLD and its derivative CXDA, which use lysine as the head group, the encapsulation method was optimized. The optimal transfection formulations of TPS/CPS and CXDA were re-formulated and explored to improve the properties of the mixed formulation for in vivo applications. This successfully established an mRNA drug encapsulation and delivery system primarily based on anionic lipids, supplemented with cationic lipids. Meanwhile, the mRNA sequence was designed with HPV E7 protein as the antigen target. By mutating the key active sites of the oncoprotein, the toxicity was reduced while retaining immunogenicity. Additionally, codon optimization and the selection of suitable non-coding regions were employed to enhance the translation efficiency of the mRNA. The optimized formulation ratio was used to encapsulate HPV E7 mRNA, successfully achieving effective treatment of mice with HPV-associated cervical cancer.

### SUMMARY

To increase the cell entry efficiency and intracellular release effectiveness of mRNA vaccines, the present disclosure provides a highly efficient and low-toxic nucleic acid drug carrier delivery system. In the present disclosure, by utilizing a mixture of anionic lipids and cationic lipids to encapsulate mRNA vaccines, more effective, safe and non-toxic in vivo mRNA delivery is achieved, thereby improving the druggability of mRNA.

In order to achieve the above object, the present disclosure adopts the following technical solutions:

The present disclosure provides a mixed nano-lipid delivery system for mRNA, wherein the delivery system is composed of an anionic and cationic mixed lipid, PEG2000-DSPE, a buffer system and mRNA;
the said anionic and cationic mixed lipid is composed of anionic nucleoside phospholipid TPS or CPS and cationic peptide lipid CLD or CLDA; and
the said buffer system is a PBS or Opti-MEM™ buffer system containing 0.1 mM Ca²⁺.

Preferably, the structural formulas of the anionic nucleoside phospholipids TPS, CPS and the cationic peptide lipids CLD, CLDA are as shown below:

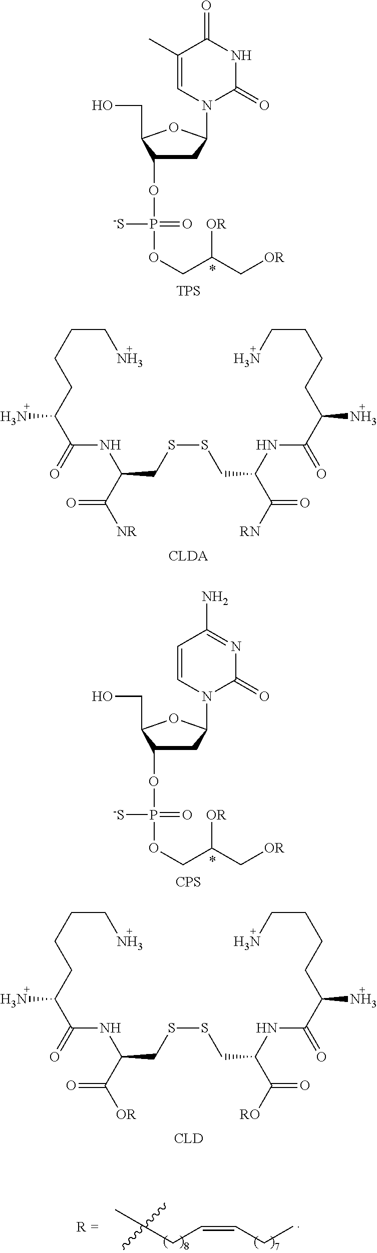

Preferably, calculated by mass ratio, TPS:CLD:mRNA=2.5:1:1, 4.5:1:1, 5.5:1:1, 6.25:1:1, 6.5:1:1, 7.5:1:1, 8.5:1:1, 4.5:3:1 or 4.5:0.5:1;
CPS:CLDA:mRNA=2.5:1:1, 3.5:1:1, 4:1:1, 4.25:1:1, 4.5:1:1, 5:1:1, 5.5:1:1, 6.5:1:1, 4.5:3:1 or 4.5:0.5:1.

Preferably, the mRNA is Firefly Luciferase mRNA, HPV E6 mutant protein mRNA or E7 mutant protein mRNA; the sequence of the HPV E6 mutant protein mRNA is shown as SEQ ID NO.1, and the sequence of the E7 mutant protein mRNA is shown as SEQ ID NO.2 or 3.

Further, the present disclosure also provides a method for preparing the mixed nano-lipid delivery system, including the following steps:
(1) weighing anionic nucleoside phospholipid TPS or CPS and cationic peptide lipid CLD or CLDA, dissolving and mixing with ethanol evenly, then lyophilizing, and dissolving with an appropriate amount of ethanol again during use to obtain a mixed lipid material;
(2) dissolving the lyophilized mixed lipid with an appropriate amount of ethanol again to obtain a liposome ethanol solution;
(3) preparation of mixed nanoliposomes of mRNA

Dissolving mRNA in nuclease-free water, adding a portion of opti-MEM or PBS containing 0.1 mM Ca²⁺, then add the liposome ethanol solution and PEG2000-DSPE ethanol solution, and finally add another portion of Opti-MEM™ or PBS containing 0.1 mM Ca²⁺, and sonicating at room temperature for 10 minutes.

Preferably, the amount of PEG2000-DSPE is 0.5% of the total molar amount of the anionic lipid and the cationic lipid.

Still further, the present disclosure provides a use of the mixed nano-lipid delivery system in the preparation of a gene therapy drug.

Preferably, the drug includes an mRNA vaccine drug, and a drug against tumors, viruses, neurodegenerative diseases, cardiovascular and cerebrovascular diseases, and diabetes.

Preferably, the drug is a drug for the prevention or treatment of tumors and other related diseases administered via one or more of routes selected from oral administration, intravenous injection, intravenous drip, intramuscular injection or subcutaneous injection.

Compared to the prior art, the present disclosure has the following advantages:
1. The present disclosure provides a mixed nano-lipid delivery system for mRNA, which is composed of an anionic lipid TPS/CPS, a cationic lipid CLD/CLDA, PEG2000-DSPE, a PBS or Opti-MEM™ buffer system containing 0.1 mM Ca²⁺, and mRNA. Anionic nucleoside lipids have a base head, which can bind to mRNA through hydrogen bonding and n-n stacking interactions. Compared to the charge interactions between cationic lipids and mRNA, this binding is more stable for in vivo applications. After injection, it enables long-term stable expression at the muscle site for over seven days, enhancing mRNA stability. Moreover, the binding based on non-electrical interactions reduces the amount of cationic lipids in the formulation. The mass ratio of cationic lipids to mRNA is only 1:1 or 0.5:1, which is much lower than the reported relative amount of cationic lipids. The present disclosure realizes efficient transfection of mRNA into cells, achieves long-term stable protein expression in vivo for more than 7 days, and has no obvious toxic and side effects, so it can be widely used in drug research related to tumor immunogene therapy.
2. The HPV E7 mRNA vaccine provided by the present disclosure can express a large amount of HPV E7 protein in cells, successfully activate humoral and cellular immunity in mice, induce the production of a large number of HPV E7-specific CD8⁺ T cells, and reduce the mortality rate of mice with HPV-related cervical cancer. Furthermore, the mRNA vaccine exhibits good safety at both cellular and animal levels with no obvious toxicity, laying a foundation for the wide clinical application of mRNA vaccines.

### BRIEF DESCRIPTION OF THE DRAWINGS

FIG. 1 shows the expression of Firefly Luciferase in HEK-293T cells delivered by TPS/CLD and CPS/CLDA liposomes for Fluc mRNA detected by a microplate chemiluminometer;

FIG. 2 shows the bioluminescent signals after protein expression of Luciferase mRNA delivered by TPS/CLD liposomes in BALB/c mice detected by an animal in vivo imaging system;

FIG. 3 shows the bioluminescent signals after protein expression of Luciferase mRNA delivered by CPS/CLDA liposomes in BALB/c mic, detected by an animal in vivo imaging system;

FIG. 4 shows the long-term bioluminescent signals after protein expression of Luciferase mRNA delivered by TPS/CLD and CPS/CLDA liposomes in BALB/c mice detected by an animal in vivo imaging system;

FIG. 5 shows the verification of HPV E7 protein expression in HEK-293T cells delivered by TPS/CLD and CPS/CLDA liposomes for the HPV E7 mRNA vaccine using Western Blot;

FIG. 6 shows the verification of HPV E7 protein expression in mouse muscles delivered by TPS/CLD and CPS/CLDA liposomes for the HPV E7 mRNA vaccine using Western Blot; and

FIG. 7 shows the tumor growth curve of cervical cancer mouse model significantly inhibited by the HPV E7 mRNA vaccine administered by intramuscular injection and the proportion of immune cells in peripheral blood of mice after treatment;

wherein, A: Dosing regimen; B: Changes in body weight during the administration period; C: Tumor growth curve; D-F: Proportion of immune cells in mouse peripheral blood.

### DETAILED DESCRIPTION

The present disclosure is further described below in conjunction with specific embodiments, and the advantages and features of the invention will become more apparent from the description of the specific embodiments. However, the embodiments are merely exemplary and do not constitute any limitation on the scope of the present disclosure. It should be understood by those skilled in the art that the details and forms of the technical solution of the present disclosure may be modified or replaced without departing from the spirit and scope of the present disclosure, but such modifications and replacements shall fall within the protection scope of the present disclosure.

### Example 1 Preparation of a Mixed Nano Delivery System for mRNA

### 1. Preparation of TPS/CLD and CPS/CLDA Mixed Lipids

(1) TPS, CLD, CPS, and CLDA were weighed according to the following mass ratios, dissolved and mixed with ethanol, then lyophilized, and dissolving with an appropriate amount of ethanol again during use;
the structural formulas of the TPS, CLD, CPS, and CLDA are shown in Formula I:

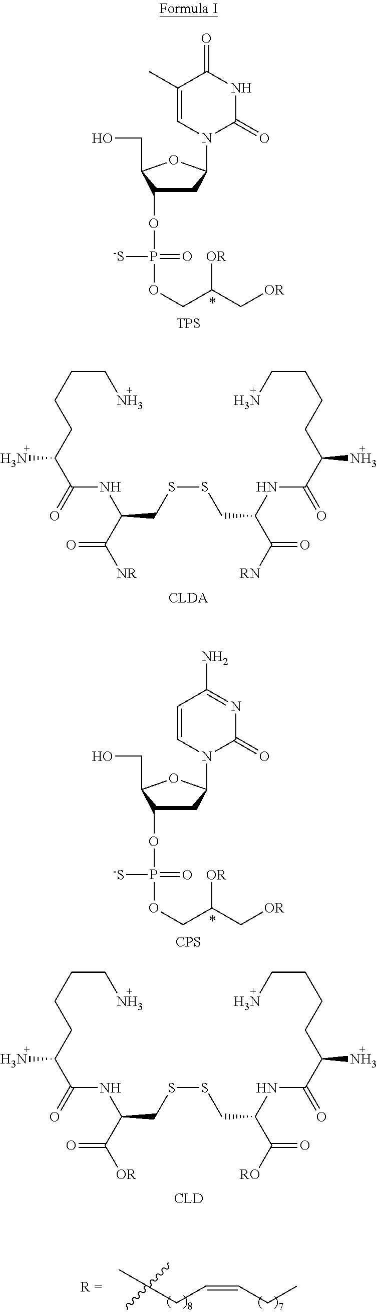

Wherein, the mass ratios of TPS to CLD were 2.5:1, 4.5:1, 5.5:1, 6.25:1, 6.5:1, 7.5:1, 8.5:1, 4.5:3, or 4.5:0.5; the mass ratios of CPS to CLDA were 2.5:1, 3.5:1, 4:1, 4.25:1, 4.5:1, 5:1, 5.5:1, 6.5:1, 4.5:3, or 4.5:0.5.
(2) The lyophilized TPS/CLD and CPS/CLDA mixed lipids were dissolved with an appropriate amount of ethanol again to obtain a liposome ethanol solution; wherein the amount of ethanol solvent was kept within 5 μL.
   2. Preparation of Mixed Nanoliposomes for mRNA

Taking a 10 μg mRNA administration dose as an example, dissolve the mRNA in nuclease-free water, take 10 μL mRNA (1 mg/mL) and add it to 40 μL opti-MEM or PBS containing 0.1 mM CaCl₂) (Mai Chen commercial reagent), then TPS/CLD or CPS/CLDA liposome ethanol solution at different mass ratios was added, and then PEG2000-DSPE ethanol solution was added, with the amount of PEG2000-DSPE at 0.5% of the total molar amount of CPS and CLDA or TPS and CLD, and the amount of ethanol solvent not exceeding 5 μL. The total volume of the formulation was made up to 100 μL by adding opti-MEM or PBS containing 0.1 mM CaCl₂). The mixture was sonicated at room temperature for 10 minutes.

The TPS/CLD mixture was mixed with mRNA at a certain mass ratio, and the mixing ratio of CPS:CLDA:mRNA was 2.5:1:1, 4.5:1:1, 5.5:1:1, 6.25:1:1, 6.5:1:1, 7.5:1:1, 8.5:1:1, 4.5:3:1, or 4.5:0.5:1.

The CPS/CLDA mixture was mixed with mRNA at a certain mass ratio, and the mixing ratio of CPS:CLDA:mRNA was 2.5:1:1, 3.5:1:1, 4:1:1, 4.25:1:1, 4.5:1:1, 5:1:1, 5.5:1:1, 6.5:1:1, 4.5:3:1, or 4.5:0.5:1.

The mRNA included Firefly Luciferase mRNA and self-designed HPV E6 and E7 mutant protein mRNAs. The sequence of the HPV E6 mutant protein mRNA is shown as SEQ ID NO.1, and the sequence of the E7 mutant protein mRNA is shown as SEQ ID NO.2 or 3.

### Example 2

This example mainly illustrated that FLuc (Firefly Luciferase) mRNA delivered by TPS/CLD and CPS/CLDA liposomes could express a large amount of Firefly Luciferase protein in cells.

### Materials and Methods

1. HEK-293T cells, FLuc mRNA, TPS/CLD (Opti-MEM) and CPS/CLDA (PBS) liposomes (Preparation method is the same as Example 1). The culture medium for HEK-293T cells was DMEM (GIBCO) medium containing 10% fetal bovine serum.
2. Detection of protein expression of FLuc mRNA delivered by TPS/CLD and CPS/CLDA liposomes in HEK-293T cells using Promega Dual-Luciferase Reporter Assay Kit. HEK-293T cells were cultured in DMEM (GIBCO) medium containing 10% fetal bovine serum in a 37° C., 5% CO₂ incubator. After observed to grow well and reach the logarithmic growth phase, the cells were seeded into 24-well plates at 1×10⁵ cells/well, and incubated in a 37° C., 5% CO₂ incubator. Transfection was performed when the cell confluence reached 80%. The experiment was divided into the cell control group, FLuc mRNA group, TPS/CLD/FLuc mRNA (TPS:CLD=6.25:1 or 6.5:1) group, CPS/CLDA/FLuc mRNA (CPS:CLDA=4.25:1) group, and Lipofectamine Messenger MAX/FLuc mRNA group. The 24-well plate was taken out, and different liposomes containing 500 ng of FLuc mRNA was added and incubated in a 37° C., 5% CO₂ incubator. After 6 hours, the medium was discarded, and cells were lysed according to the instructions of the Dual-Luciferase Assay Kit, then the lysate was transferred to a 96-well white plate, and detected using a microplate chemiluminometer by adding 50 μL of lysate and 50 μL of luciferase substrate to each well.

### Results

The CPS/CLDA group showed high Firefly luciferase protein expression at 6 h, 24 h and 48 h (FIG. 1).

In summary, the FLuc mRNA delivered by CPS/CLDA liposomes could express a large amount of Firefly luciferase in cells, and this delivery system could efficiently and stably deliver mRNA into cells for stable expression.

### Example 3

This example mainly illustrated that the mRNA delivered by TPS/CLD and CPS/CLDA liposomes could express proteins in BALB/c mice.

### Materials and Methods

1. Male BALB/c mice, SPF grade, 25 g each, provided by Beijing Vital River Laboratory Animal Technology Co., Ltd.; Luciferase mRNA (APExBIO).

### Reagent Preparation:

TPS/CLD/Luciferase mRNA complex and CPS/CLDA/Luciferase mRNA complex (prepared with 3 μg Luciferase mRNA per mouse): Luciferase mRNA was dissolved in nuclease-free water to a concentration of 1 μg/μL. Then, 9.9 μL of Luciferase mRNA was added to opti-MEM or PBS containing 0.1 mM CaCl₂), then TPS/CLD or CPS/CLDA liposome solutions at different mass ratios (Preparation method is the same as Example 1) was added. PEG2000-DSPE ethanol solution was added, with the amount of PEG2000-DSPE equal to 0.5% of the total molar amount of CPS and CLDA or TPS and CLD. The volume of ethanol solvent was kept within 5 μL. Finally, opti-MEM or PBS was added to make up to 100 μL, and the mixture was sonicated for 10 minutes.
2. Experimental grouping: Blank group, TPS/CLD/Luciferase mRNA complex group, CPS/CLDA/Luciferase mRNA complex group, and Lipofectamine Messenger MAX/FLuc mRNA complex group. Mice in the three complex groups were injected with 100 μL of mRNA-containing liposome solution by intramuscular injection, and the mice in the blank group were injected with an equivalent dose of normal saline. At 6 h, 12 h, 24 h, 48 h, 100 h, and 171 h, these animals received intraperitoneal injection of 15 mg/mL D-Luciferin (40901ES03), and 10 minutes later, anesthetized with gas for 3 minutes, and finally, the in vivo bioluminescent signals in mice were detected using an in vivo imaging system for small animals.

### Results

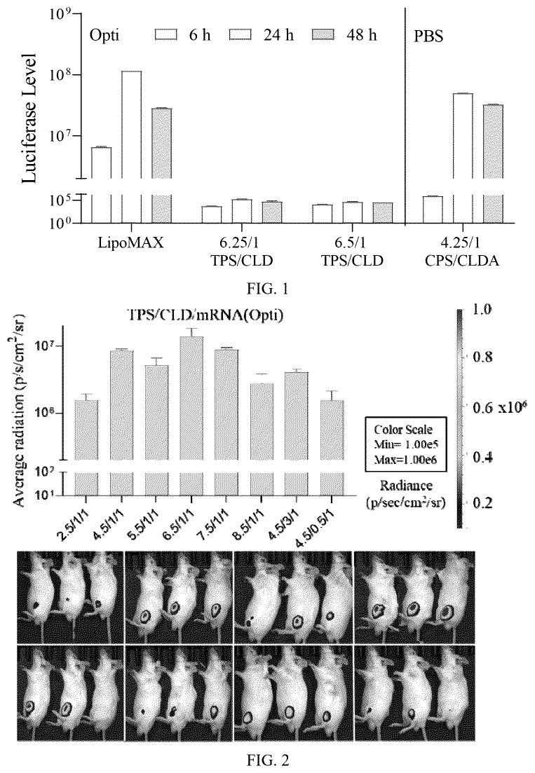

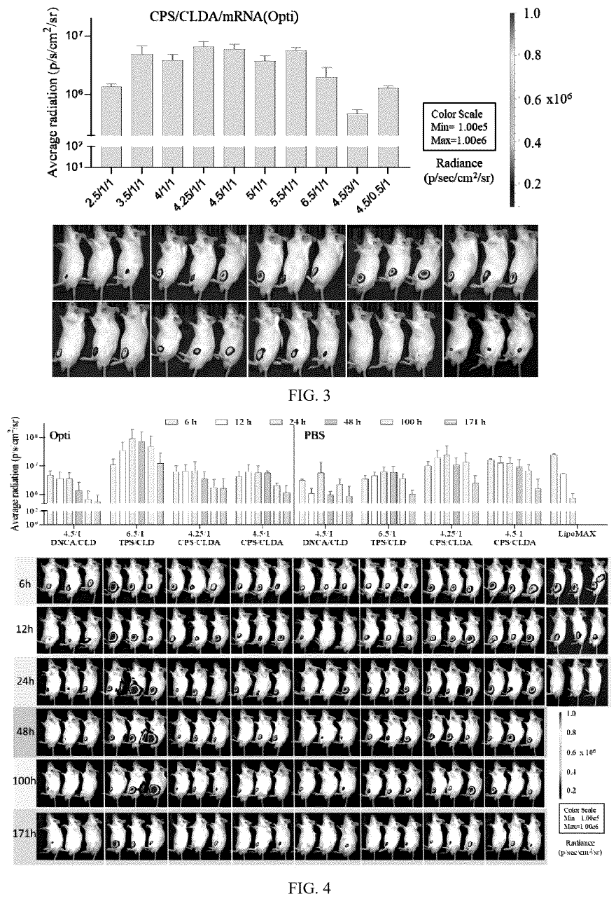

The in vivo imaging system for small animals showed that mice in the TPS/CLD/Luciferase mRNA complex group and CPS/CLDA/Luciferase mRNA complex group exhibited obvious bioluminescent signals at 6 h. Moreover, when the mass ratio of TPS/CLD/mRNA was 6.5/1/1 (FIG. 2) and the mass ratio of CPS/CLDA/mRNA was 4.25/1/1 (FIG. 3), the highest delivery efficiency was achieved. By extending the imaging time, bioluminescent signals were detected at 6 h, 12 h, 24 h, 48 h, 100 h, and 171 h (FIG. 4). These results indicated that mRNA delivered by TPS/CLD and CPS/CLDA liposomes could express luciferase protein efficiently and stably in BALB/c mice, with the longest duration of more than seven days.

### Example 4

This example mainly illustrated the use of TPS/CLD and CPS/CLDA/liposomes in delivering HPV E7 mRNA vaccines.

### Materials and Methods

1. HEK-293T cells, TPS/CLD and CPS/CLDA liposomes (prepared by the method in Example 1), male BALB/c mice, SPF grade, 25 g each, provided by Beijing Vital River Laboratory Animal Technology Co., Ltd. The self-designed HPV E7 mRNA 7-1 (E7-1, SEQ ID NO.2) and 7-2 (E7-2, SEQ ID NO.3) were synthesized by APExBIO upon commission.
2. Determination of the expression of antigen proteins delivered by TPS/CLD and CPS/CLDA encapsulated HPV E7 mRNA vaccines in cells using Western Blot.
(1) HEK-293T cells were cultured in DMEM (GIBCO) medium containing 10% fetal bovine serum in a 37° C., 5% CO₂ incubator. The cells were observed to be in good growth condition. After culturing to the logarithmic growth phase, the cells were seeded into 6-well plates at 1×10⁶ cells per well and incubated at 37° C. with 5% CO₂ in an incubator. After observed to grow well and reach the logarithmic growth phase, the cells were seeded into 6-well plates at 1×10⁶ cells/well, and incubated in a 37° C., 5% CO₂ incubator. Transfection was performed when the cell confluence reached 80%. The experiment was divided into the cell control group, CPS/CLDA/HPV E7-1 mRNA group (4.25/1/1, 1 μg) or TPS/CLD/HPV E7-1 mRNA group (6.25/1/1, 1 μg), and CPS/CLDA/HPV E7-2 mRNA group (4.25/1/1, 1 μg) or TPS/CLD/HPV E7-2 mRNA group (6.25/1/1, 1 μg). The 24-well plate was taken out, and the liposomes containing 1 μg of HPV E7-1 or 7-2 mRNA were added to each well, and incubated in a 37° C., 5% CO₂ incubator for 24 hours.
(2) The total proteins from cells in each well were extracted for Western Blot.
\textcircled{1} 10% SDS-polyacrylamide gel electrophoresis (electrophoresis at 140 V for 60 min) was performed. After electrophoresis, the gel was taken out and the desired portion of the gel was retained based on the position of the protein marker; the PVDF membrane and filter paper were cut according to the gel size, and the PVDF membrane was activated in methanol for 1 minute, then the membrane and filter paper were placed in the transfer buffer for 10 min. The filter paper, PVDF membrane, gel, and filter paper were placed on the transfer apparatus from bottom to top. The current was set according to the size of the filter paper, with the duration of 1.5-2 h. After completion of transfer, the membrane was placed in 50 mL of 5% skimmed milk, incubated at 40 r/min for 40 min with shaking, then blocked overnight. After blocking, the membrane was transferred to HPV E7 primary antibody (Thermo Scientific, 1:1000), and incubated at 40 r/min for 2 h at room temperature with shaking. The membrane was washed with 1× blocking buffer at 80 r/min for three times, 15 min each time. The washed membrane was transferred to the secondary antibody (Proteintech, 1:2000) and incubated with shaking at room temperature for 1 h; the membrane was washed with 1× blocking solution at 80 r/min for 3 times (5 min, 5 min, and 20 min, respectively). The PVDF membrane was placed in an imager for development.
\textcircled{2} Internal reference quantification: The PVDF membrane was washed with 1× blocking buffer at 80 r/min for 15 min; the washed membrane was placed into a membrane regeneration solution for 30 min. After membrane regeneration, the membrane was washed with 1× blocking buffer at 80 r/min for 15 min. The membrane was transferred to the anti-α-actin primary antibody (Proteintech, 1:50000) and incubated overnight; the membrane was washed 3 times with 1× blocking buffer at 80 r/min for 15 min; then the washed membrane was transferred to the secondary antibody (Proteintech, 1:2000) and incubated with shaking at room temperature for 1 h; the membrane was washed 3 times with 1× blocking buffer at 80 r/min for 15 min; and then the PVDF membrane was placed in an imager for development.
3. 3×10⁵ mouse TC-1 tumor cells were injected into the right axilla of female C57BL/6 mice to establish a subcutaneous cervical cancer model. The treatment began when the tumor volume reached approximately 50 mm² after inoculation. The first dose of HPV E7 mRNA (10 μg/mouse) encapsulated by TPS/CLD or CPS/CLDA was administered via intramuscular injection into the ipsilateral thigh, followed by a second booster injection seven days later to enhance the immune effect.

### Results

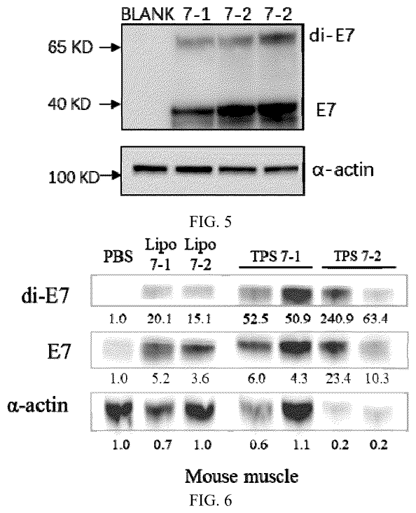

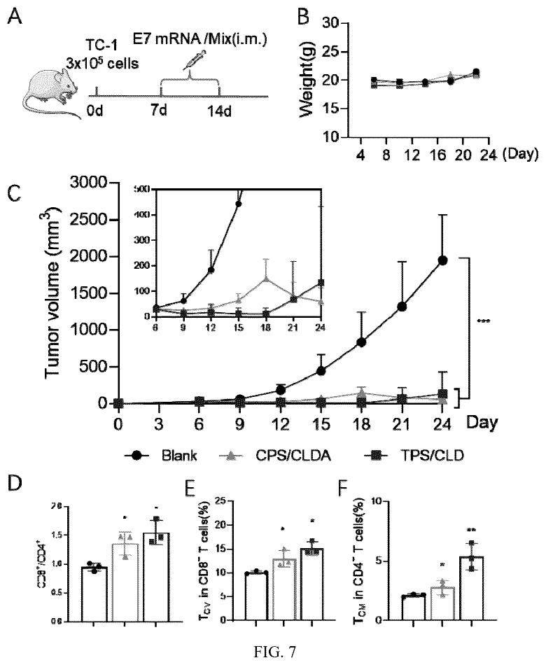

Western Blot experiments demonstrated that the HPV E7 mRNA vaccines delivered by CPS/CLDA liposomes could express HPV E7 protein both intracellularly and in mouse muscles (FIG. 5 and FIG. 6). The HPV E7 mRNA vaccines significantly inhibited tumor growth in the mouse subcutaneous cervical cancer model (FIG. 7). Approximately 83% (5/6) and 66% (4/6) of mice in the TPS/CLD and CPS/CLDA encapsulate groups achieved complete tumor regression, respectively. Additionally, the CD8⁺/CD4⁺ ratio and the proportion of TCM cells in both cell types were significantly increased, suggesting good immune response and long-term immune memory; the body weight of mice in each group increased steadily during treatment, with no obvious weight loss, indicating good drug safety.

The information displayed and described in detail herein is sufficient to achieve the aforementioned objectives of the present disclosure. Therefore, the preferred embodiments of the present disclosure represent the subject matter of the invention, which is broadly covered by the present disclosure. The scope of the present disclosure fully encompasses other embodiments that are obvious to those skilled in the art. Therefore, the scope of the present disclosure is not limited by anything other than the appended claims. Unless explicitly stated otherwise, the singular form of an element does not denote “one and only one” but “one or more”. To those skilled in the art, all known structural, compositional, and functional equivalents of the above-described preferred embodiments and additional embodiments are hereby incorporated by reference and are intended to be encompassed by the claims of the present disclosure.

Furthermore, it is not necessary to have a specific device or method to address each problem solved by the present disclosure, as they are all encompassed within the claims of the present disclosure. Additionally, no parts, components, or method steps disclosed in the present disclosure, whether explicitly described in the claims or not, are dedicated to the public. However, it will be apparent to those skilled in the art that various changes and modifications can be made in form, reagents, and synthetic details without departing from the spirit and scope of the present disclosure as set forth in the appended claims.

1. A mixed nano-lipid delivery system for mRNA, wherein the delivery system is composed of an anionic and cationic mixed lipid, PEG2000-DSPE, a buffer system and mRNA;
the said anionic and cationic mixed lipid is composed of anionic nucleoside phospholipid TPS or CPS and cationic peptide lipid CLD or CLDA; and
The said buffer system is a PBS or Opti-MEM™ buffer system containing 0.1 mM Ca²⁺.

2. The mixed nano-lipid delivery system according to claim 1, wherein the structural formulas of the anionic nucleoside phospholipids TPS, CPS and the cationic peptide lipids CLD, CLDA are as shown below:

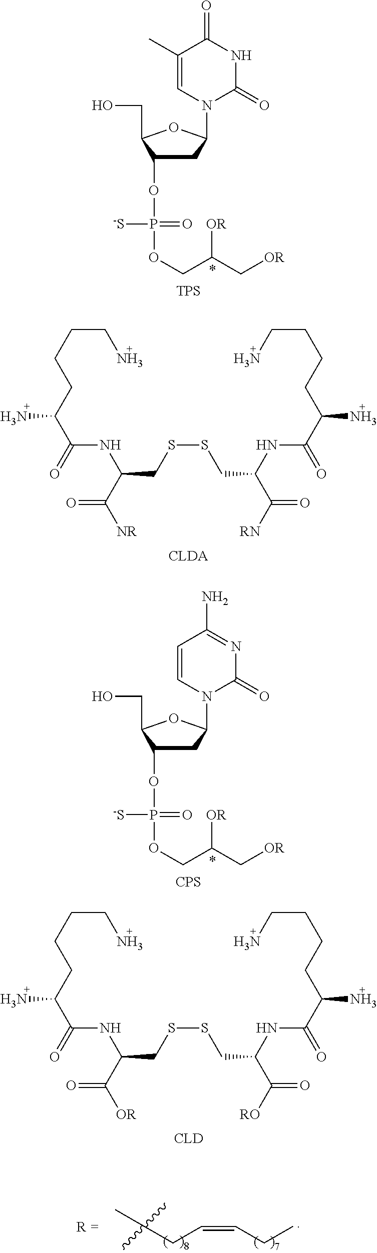

3. The mixed nano-lipid delivery system according to claim 1, wherein, calculated by mass ratio, TPS:CLD:mRNA=2.5:1:1, 4.5:1:1, 5.5:1:1, 6.25:1:1, 6.5:1:1, 7.5:1:1, 8.5:1:1, 4.5:3:1 or 4.5:0.5:1;
CPS:CLDA:mRNA=2.5:1:1, 3.5:1:1, 4:1:1, 4.25:1:1, 4.5:1:1, 5:1:1, 5.5:1:1, 6.5:1:1, 4.5:3:1 or 4.5:0.5:1.

4. The mixed nano-lipid delivery system according to claim 1, wherein the mRNA is Firefly Luciferase mRNA, HPV E6 mutant protein mRNA or E7 mutant protein mRNA; the sequence of the HPV E6 mutant protein mRNA is shown as SEQ ID NO.1, and the sequence of the E7 mutant protein mRNA is shown as SEQ ID NO.2 or 3.

5. A method for preparing the mixed nano-lipid delivery system according to claim 1, comprising the following steps:
(1) weighing anionic nucleoside phospholipid TPS or CPS and cationic peptide lipid CLD or CLDA, dissolving and mixing with ethanol evenly, then lyophilizing, and dissolving with an appropriate amount of ethanol again during use to obtain a mixed lipid material;
(2) dissolving with an appropriate amount of ethanol again during use to obtain a liposome ethanol solution;
(3) preparation of mixed nanoliposomes of mRNA
dissolving mRNA in nuclease-free water, adding a portion of opti-MEM or PBS containing 0.1 mM Ca²⁺, then add the liposome ethanol solution and PEG2000-DSPE ethanol solution, and finally add another portion of Opti-MEM™ or PBS containing 0.1 mM Ca²⁺, and sonicating at room temperature for 10 minutes.

6. The method according to claim 5, wherein the amount of PEG2000-DSPE is 0.5% of the total molar amount of the anionic lipid and the cationic lipid.

7. Use of the mixed nano-lipid delivery system of claim 1 in the preparation of a gene therapy drug.

8. The use according to claim 7, wherein the drug comprises an mRNA vaccine drug, and a drug against tumors, viruses, neurodegenerative diseases, cardiovascular and cerebrovascular diseases or diabetes.

9. The use according to claim 8, wherein the drug is a drug for the prevention or treatment of tumors and other related diseases administered via one or more of routes selected from oral administration, intravenous injection, intravenous drip, intramuscular injection or subcutaneous injection.

10. A method for preparing the mixed nano-lipid delivery system according to claim 2, comprising the following steps:
(1) weighing anionic nucleoside phospholipid TPS or CPS and cationic peptide lipid CLD or CLDA, dissolving and mixing with ethanol evenly, then lyophilizing, and dissolving with an appropriate amount of ethanol again during use to obtain a mixed lipid material;
(2) dissolving with an appropriate amount of ethanol again during use to obtain a liposome ethanol solution;
(3) preparation of mixed nanoliposomes of mRNA
dissolving mRNA in nuclease-free water, adding a portion of opti-MEM or PBS containing 0.1 mM Ca²⁺, then add the liposome ethanol solution and PEG2000-DSPE ethanol solution, and finally add another portion of Opti-MEM™ or PBS containing 0.1 mM Ca²⁺, and sonicating at room temperature for 10 minutes.

11. A method for preparing the mixed nano-lipid delivery system according to claim 3, comprising the following steps:
(1) weighing anionic nucleoside phospholipid TPS or CPS and cationic peptide lipid CLD or CLDA, dissolving and mixing with ethanol evenly, then lyophilizing, and dissolving with an appropriate amount of ethanol again during use to obtain a mixed lipid material;
(2) dissolving with an appropriate amount of ethanol again during use to obtain a liposome ethanol solution;
(3) preparation of mixed nanoliposomes of mRNA
dissolving mRNA in nuclease-free water, adding a portion of opti-MEM or PBS containing 0.1 mM Ca²⁺, then add the liposome ethanol solution and PEG2000-DSPE ethanol solution, and finally add another portion of Opti-MEM™ or PBS containing 0.1 mM Ca²⁺, and sonicating at room temperature for 10 minutes.

12. A method for preparing the mixed nano-lipid delivery system according to claim 4, comprising the following steps:
(1) weighing anionic nucleoside phospholipid TPS or CPS and cationic peptide lipid CLD or CLDA, dissolving and mixing with ethanol evenly, then lyophilizing, and dissolving with an appropriate amount of ethanol again during use to obtain a mixed lipid material;
(2) dissolving with an appropriate amount of ethanol again during use to obtain a liposome ethanol solution;
(3) preparation of mixed nanoliposomes of mRNA
dissolving mRNA in nuclease-free water, adding a portion of opti-MEM or PBS containing 0.1 mM Ca²⁺, then add the liposome ethanol solution and PEG2000-DSPE ethanol solution, and finally add another portion of Opti-MEM™ or PBS containing 0.1 mM Ca²⁺, and sonicating at room temperature for 10 minutes.

13. Use of the mixed nano-lipid delivery system of claim 2 in the preparation of a gene therapy drug.

14. Use of the mixed nano-lipid delivery system of claim 3 in the preparation of a gene therapy drug.

15. Use of the mixed nano-lipid delivery system of claim 4 in the preparation of a gene therapy drug.
